(19) United States
(12) Patent Application Publication
(10) Pub. No.: US 2023/0398254 A1
(43) Pub. Date: Dec. 14, 2023

(54) METHODS AND DEVICES FOR MOBILE HAND CLEANING AND SANITATION

(71) Applicant: Thomas Eric Asacker, Exeter, NH (US)

(72) Inventor: Thomas Eric Asacker, Exeter, NH (US)

(21) Appl. No.: 18/326,865

(22) Filed: May 31, 2023

Related U.S. Application Data
(60) Provisional application No. 63/351,226, filed on Jun. 10, 2022.

(51) Int. Cl. A61L 2/26; A61L 2/00

(52) U.S. Cl. CPC A61L 2/26; A61L 2/0088; A61L 2202/16

(57) ABSTRACT

A mobile system and method for sterilizing hands includes a reservoir filled with sterilizing fluid. The reservoir is inside of an outer shell that is adapted for a user to insert hands into the reservoir to sterilize the hands. A pair of valves can help to wipe excess fluid from the hands and retain the fluid within the reservoir. A pair of end cuffs can help to remove excess fluid from the hands of the user. The mobile system for sterilizing hands can be worn by the user, so that the user can insert hands, sterilize the hands, and remove the hands from the sterilizer without needing to travel to a sink or other infrastructure.

### RELATED APPLICATIONS

This claims the benefit of U.S. Provisional Application Ser. No. 63/351,226, filed Jun. 10, 2022, entitled METHODS AND DEVICES FOR MOBILE HAND CLEANING AND SANITATION, the entire disclosure of which is herein incorporated by reference.

### FIELD OF THE INVENTION

This application relates to hand sanitation, and more particularly, to mobile hand sanitation in various environments including healthcare, homecare, daycare, schools, and restaurants.

### BACKGROUND OF THE INVENTION

Hand sanitation can be of utmost importance in a variety of settings, including hospitals, nursing homes, long-term care, in-home care, restaurants, schools, day care, and various other settings. Scientific advancements have allowed mankind to understand the role of bacteria and viruses in causing human illness, and further scientific advancements have helped mankind learn how to kill those illness-causing bacteria and viruses. As used herein, the word “germs” will refer to bacteria and/or viruses, including bacteria and/or viruses that can lead to human illness. The word “germs” also includes various drug-resistant bacteria, including MRSA (Methicillin-Resistant Staphylococcus Aureus). Good hand hygiene is one of the best ways to prevent the spread of germs, including drug-resistant germs. However, despite the clear advantages to keeping hands sanitary, many people who work in settings such as healthcare and restaurants do not clean their hands as often as is necessary for good sanitation.

There may be a number of reasons for poor sanitation, despite knowing the importance of keeping clean hands. As an example, a busy hospital worker may have more work to do than can be accomplished in a given shift, and may get busy and forget to walk to a sink or other sanitization units to wash or disinfect hands after each patient interaction. In many cases this may be unconscious non-compliance with procedures. Although this may allow them to accomplish more tasks in a given period of time, it can also lead to unsanitary conditions, including germs being spread freely from one patient to the next. As another example, a daycare worker may have a large number of small children to keep track of, and that daycare worker may not be able to take the time away from their charges to find a sink and wash hands after each interaction with a child, especially in the case of children who require constant supervision.

Many healthcare settings have various posters, reminders, reporting programs, etc., designed specifically to increase hand-washing compliance. However, even with numerous programs in place, hand-washing compliance is rarely perfect, resulting in the spread of germs despite the best intentions of hospitals or other institutions. Furthermore, patients, families, or other users of healthcare services do not know whether a healthcare provider has followed the guidance to wash hands before entering that patient's room. A patient may not want to insult a healthcare provider by asking them if they washed their hands before entering the room, and may not trust the answer if they do ask.

It would be desirable to have a hand sanitation system that is convenient to use and present at every location where it is needed, when it is needed. It would be further desirable to have a hand sanitation system that provides a visual cue as a reminder to perform the hand hygiene routine, and which enables a patient or patient's family to know the hand sanitation status of a healthcare provider without needing to ask.

### SUMMARY OF THE INVENTION

As described herein, this disclosure overcomes disadvantages of the prior art by providing a mobile and portable hand sanitation system. The mobile and portable hand sanitization system can be enclosed in a pouch, outer shell, or other vessel, that can be positioned around a user's torso, or in various strategic locations around a building. The mobile hand sanitization system can contain a hand sanitizing fluid or other sanitizing solution that can be inside of an inner reservoir of the containment system. The system can include valves to contain the sanitizing fluid, and can include end cuffs that can help to remove excess fluid from the hands as the hands are removed from the hand sanitization system.

In addition to the sanitizing fluid, the system can include various coaters, agitators, and/or scrubbers. As used herein, a coating enhancer can assist with applying sanitizing fluid on the hands, in addition to providing friction and scrubbing capabilities. The coating enhancer can include a large number of small balls, various brushes, or other objects designed to provide thorough coating and additional friction contact with the skin of the user's hand to assist in scrubbing and/or coating the hands of the user.

A user can insert hands into the pouch, through the end cuff, through the valve, and into the reservoir of sanitizing fluid. The user can then rub the hands together and/or rub the hands on the various coating and/or friction enhancing surfaces inside the containment system. After rubbing the hands with the sanitizing fluid, the user can pull the hands out through the valve which can constrict around the hands and wipe off excess fluids. The user can also pull the hands through the end cuffs, which can contain brushes, absorbent materials, wipers, or other tools for removing excess fluids. The user can then remove the sanitized hands from the sanitizing system and proceed to other tasks while having sanitized hands. Other people in the immediate area can see the hands being removed from the sanitizer and know that the user's hands have been sanitized while they were in the sanitizing system.

The mobile hand sanitizing system can provide hand-sanitizing opportunities any time and at nearly any location without the need for running water or other infrastructure. The mobile hand sanitizing system can also allow for quick, easy, and convenient hand sanitization without requiring a user to grab anything or interact with anything except the hand sanitizing system that can be positioned for a user to quickly and easily insert hands. The mobile hand sanitizing system can also allow other people in the immediate area to know that the user's hands have been cleaned and sanitized.

In an embodiment, a mobile hand sanitization system can include an outer shell having an opening at each of two ends of the outer shell, and a reservoir within the outer shell, where the reservoir is adapted to be filled with sanitizing fluid. The mobile hand sanitizing system can have an end cuff lined with absorbent material, wherein the end cuff can be adapted to wipe excess sanitizing fluid from the hands of the user. The mobile hand sanitizing system can have an end cuff lined with brushes, wherein the end cuff can be adapted to wipe sanitizing fluid from the hands of the user. The mobile hand sanitizing system can have at least one valve at each of the openings, and the valves can be adapted to wipe sanitizing fluid from hands of a user into the reservoir. The mobile hand sanitizing system can have one or more coating enhancers within the reservoir, and the coating enhancers can include various filaments. The mobile hand sanitizing system can include a disposable and replaceable bladder, and the bladder can contain a sanitizing solution. The mobile hand sanitizing system can include one or more coating enhancers within the reservoir. The coating enhancers can include filaments of rubber attached to the sidewalls of the reservoir. The coating enhancers can include free floating balls within the reservoir. The free-floating balls can have filaments of rubber.

In an embodiment, a disposable bladder for use in a mobile hand sanitizing system can include an outer sidewall adapted to contain sanitizing fluid, and coating enhancers within the disposable bladder. The coating enhancers can include filaments on the bladder walls, and the filament can extend into the sanitizing fluid. The coating enhancers can include free-floating balls within the sanitizing fluid. The free-floating balls can have filaments extending outward from the balls.

In an embodiment, a method of hand sanitization can include inserting hands into a mobile hand sanitization system that can include a reservoir of sanitizing fluid, moving the hands within the hand sanitizing solution, and pulling the hands out of the reservoir. Pulling the hands out of the reservoir can include pulling the hands through a valve, the valve adapted to wipe excess fluid off of the hands and retain the fluid within the reservoir. The method can include pulling the hands through end cuffs, and the end cuffs can include absorbent materials adapted to remove fluid from the hands of the user. The method can include removing the end cuffs after they become saturated with fluid, and replacing the end cuffs with different end cuffs. The method can include pulling the hands through end cuffs, and the end cuffs can include brushes adapted to remove fluid from the hands of the user.

### BRIEF DESCRIPTION OF THE DRAWINGS

Several embodiments of are illustrated by these accompanying drawings, of which:

FIG. 1 is a perspective view of a mobile hand sanitizing system, according to an illustrative embodiment;

FIG. 2 is a partially cut away view of the mobile hand sanitizing system, showing inner workings, according to an illustrative embodiment;

FIG. 3A is a perspective view of a bead-style coating enhancer, according to an illustrative embodiment;

FIG. 3B is a perspective view of a thick filament type coating enhancer, according to an illustrative embodiment

FIG. 3C is a perspective view of a thin filament type coating enhancer, according to an illustrative embodiment;

FIG. 3D is a perspective view of a brush-type coating enhancer, according to an illustrative embodiment;

FIG. 4 is a perspective view of a mobile hand sanitizing system worn around the waist of a user, according to an illustrative embodiment; and

FIG. 5 is a diagram showing a method of mobile hand sanitation, according to an illustrative embodiment.

### DETAILED DESCRIPTION

A mobile and portable hand sanitization system can be a mobile and portable device used to sanitize a user's hands. A mobile and portable hand sanitization system can also be referred to as a mobile hand sanitization system, mobile hand sanitizing system, mobile hand sanitizer, a portable hand sanitization system, portable hand sanitizing system, a portable hand sanitizer, hand sanitizer, hand cleaner, etc. It should be clear that these terms can be used interchangeably, and refer to a mobile and portable hand sanitization system that can be used to sanitize a user's hands.

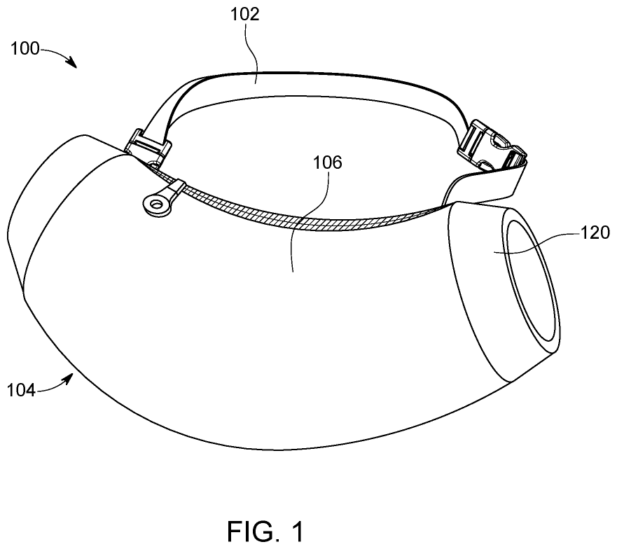

FIG. 1 is a perspective view of a mobile hand sanitizing system, according to an illustrative embodiment. A mobile hand sanitizing system 100 can be small, flexible, and lightweight, so that a user can easily carry the device or can wear the device, for example, around the waist or over the shoulder(s). In various embodiments, the mobile hand sanitizing system 100 can include a strap 102 that can hold the device in place to be worn by the user. The mobile hand sanitizing system 100 can have a body 104 that includes an outer shell 106. Outer shell 106 can be made from a rigid material and/or various fluid-resistant flexible materials. The materials used for the outer shell 106 of the mobile hand sanitizing system can include various fluid resistant and antimicrobial materials, and can include fluid resistant fabrics and/or various layers of different materials that can be combined to create a flexible and protective outer shell to contain the device. In various embodiments, an outer shell can include materials that are flexible while also sufficiently stiff to maintain a desired shape.

Institutions such as hospitals and other healthcare institutions can provide various insignias and/or information on the outside of the mobile hand sanitizing system to inform patients and patients' families of the nature of the device. Information on the front of the mobile hand sanitizing system can alert patients and patient's families as to the nature of the device, so that the patients and families will be comforted by seeing healthcare workers using the device.

A hand sanitizing system can have an inner reservoir of sanitizing fluid that can be any one or more of various liquids, gases, gels, foams, sanitizing solutions, alcohols, or other fluids capable of killing germs. The sanitizing fluid of the hand sanitizing system can be contained within an inner bladder. The inner bladder can be filled with a sanitizing fluid and adapted for the user to insert their hands into. The mobile hand sanitizing system can have two openings for a user to insert both hands. In various embodiments, the mobile hand sanitizing system can be somewhat tube-shaped, with an opening at either end for each hand. Each opening can include an end cuff 120 that can help to remove excess sanitizing fluid from the hands of the user as the user removes the hands from the device. The end cuff 120 can also add structure to the body 104, and can tactilely steer the hands of the user towards fluid reservoir of the mobile hand sanitizing system. In various embodiments, the end cuff can be lined with absorbent materials, various brushes, scrapers, and/or other materials to aid in removing excess sanitizing fluid from the hands of the user as the user retracts the hands out of the mobile sanitizer. In various embodiments, the end cuff 120 can be removable, replaceable, washable, and/or disposable. After an end cuff has absorbed excess sanitizing fluid, the end cuff may gradually lose capacity to absorb fluid as more and more fluid has already been absorbed. If the end cuff becomes too saturated to be effective at removing excess fluid, the end cuff can be removed and a replacement end cuff can be installed on the mobile hand sanitizing system. Replacing the cuff as it loses capacity to remove sanitizing fluid can allow the user to continue using the same device without needing to replace the entire hand sanitizing system.

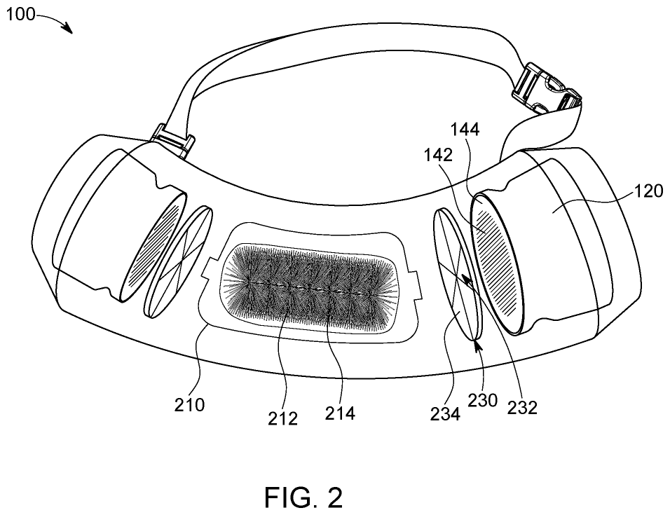

FIG. 2 is a partially cut away view of the mobile hand sanitizing system, showing inner workings, according to an illustrative embodiment. A mobile hand sanitizing system 100 can have an inner reservoir of hand sanitizing fluid 212. In various embodiments, the reservoir can be within an inner bladder 210, so that the sanitizing fluid can be contained within that inner bladder 210. The sanitizing fluid 212 can be various liquids, gases, gels, foams, sanitizing solutions, alcohols, or other fluids capable of killing germs. The reservoir can also contain various coating enhancers 214. Coating enhancers 214 can include various balls, beads, or other materials capable of increasing the friction on the hands and/or helping to coat the hands of the user as the user moves the hands inside the reservoir. Coating enhancers can also be referred to as friction enhancers or contact enhancers, however, it should be clear that the terms coating enhancer and friction enhancer can refer to the same component, and it should be clear that the coating enhancer can enhance the thoroughness of the coating as well as assist in applying friction.

In various embodiments, the reservoir can be filled with sanitizing fluid. In various embodiments, the reservoir can be partially filled with sanitizing fluid. In various embodiments, the reservoir can have a small quantity of sanitizing fluid that can coat the surfaces of the reservoir walls and/or the coating enhancers, and sanitizing fluid can be wiped onto the hands of the user as the user inserts the hands into the reservoir and moves the hands around. The coating enhancers can allow the hand sanitizing system to be effective without requiring the reservoir to be filled with a heavy fluid. A small quantity of fluid can effectively coat the hands of the user while keeping the weight of the system to a minimum. In various embodiments, a small quantity of fluid can pool in the bottom of the reservoir, and that small quantity of fluid can then coat the coating enhancers between uses, so that when the user inserts hands into the reservoir the coating enhancers are coated with fluid that can then coat and sterilize the hands of the user.

A user can move their hands inside the reservoir, and the coating enhancers 214 can rub the sanitizing fluid 212 on the hands of the user. The reservoir can be designed to be emptied, cleaned, sanitized, sterilized, and refilled for repeated usages. Because the reservoir can be emptied, cleaned, and refilled, a user can use the device without the need for concern about any debris that may be deposited within the reservoir during normal usage.

In various embodiments, a bladder 210 can be refillable, washable and/or otherwise cleanable. In various embodiments, the bladder 210 or can be a single-use disposable bladder. A disposable bladder may come pre-filled with various sanitizing fluids and/or coating enhancers 214. In various embodiments, coating enhancers 214 can also be single-use, disposable, and replaceable, and the coating enhancers 214 may come pre-loaded in the disposable bladder 210 so that the bladder, the coating enhancers, and any fluid can be replaced at the same time with a single replacement bladder.

The mobile hand sanitizing system can also include a valve 230 at either end of the reservoir. The valve can include a fluid resistant dam 234 with a stretchable or expandable opening 232. The user can insert hands through the expandable opening 232, and then after interacting with the sanitation fluid, the user can retract the hands back out through the expandable opening. As the expandable opening 232 stretches around the hands of the user, excess fluid 212 is wiped off of the hands and is retained inside of the mobile hand sanitizing system.

When the mobile hand sanitizing system 100 is not in use, the valves 230 are in a closed or nearly closed conformation to keep the fluid 212 from exiting the mobile hand sainting system 100 as the user walks or performs other tasks. As the user pushes hands through the valves 230, the valves are stretched to an open conformation. In the open conformation, the valves are constricted around the hands or wrists of the user. The valves can remain tight around the hands of the user as the user removes the hands, so that the excess fluid is wiped off of the hands as they are removed from the mobile hand sanitizing system 100.

End cuffs 120 can further assist in removing excess fluid 212. The end cuffs can be lined with various brushes or wipers to further assist in scraping excess fluid off of the hands of the user. The end cuffs can be lined with absorbent materials 142 and/or brushes 144 that can wipe and absorb excess fluid from the hands of the user. The term brushes can include various fabrics or other materials. After the user has pulled the hands through the valves, thereby wiping away excess fluid, and then pulled the hands through the end cuffs, thereby further removing remaining fluids, the user's hands can be rid of excess fluid. The user can then rub the two hands together outside of the mobile hand sanitation system to further dry the hands in preparation for the activities to be performed by the user with the freshly sanitized hands.

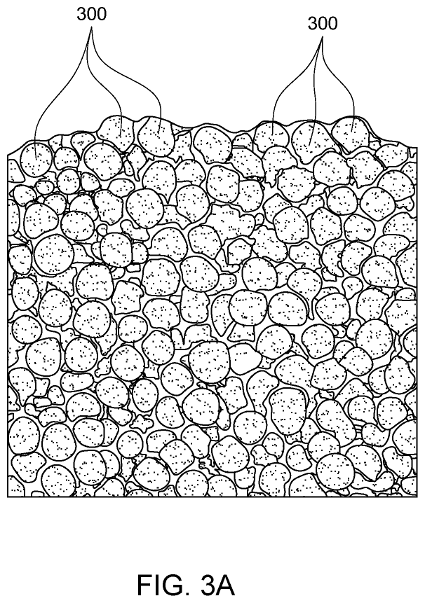

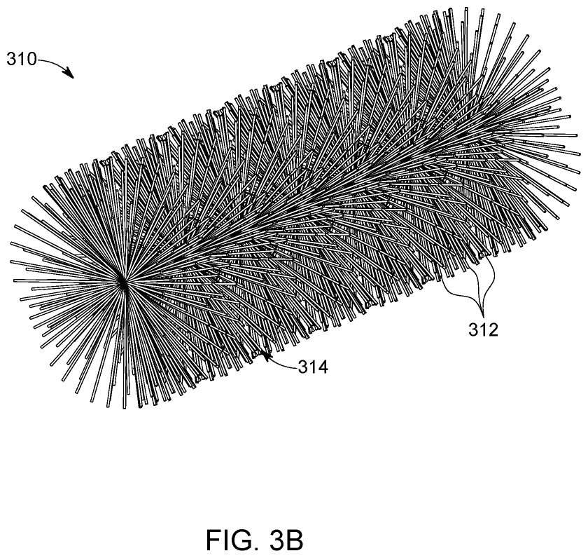

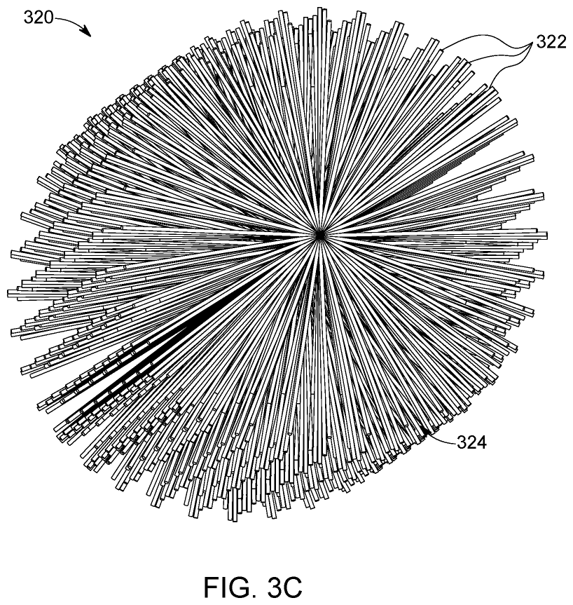

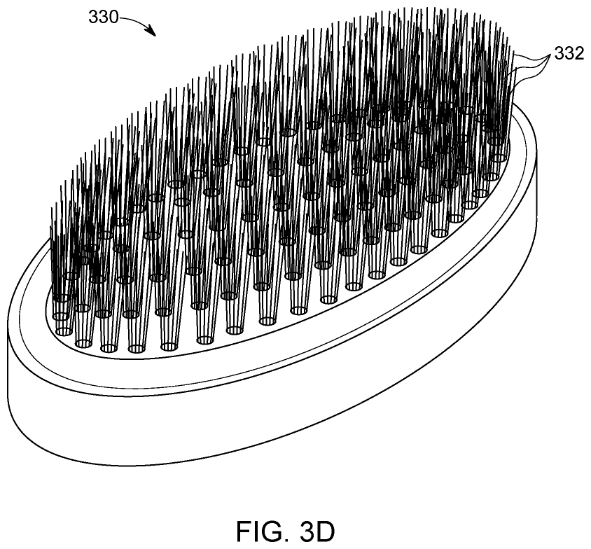

Various embodiments can include various types of coating enhancers 214, and some embodiments may use more than one coating enhancer and/or combine features of various coating enhancers described herein. The coating enhancers 214 can rub the sanitizing fluid 212 against the hands of the user to assist the sanitizing fluid in killing germs on the hands of the user. FIG. 3A is a perspective view of a bead-style coating enhancer 300, according to an illustrative embodiment. A coating enhancer can include a number of small beads within the sanitizing fluid 212. The small beads can rub against the hands of the user to assist the sanitizing fluid 212 in killing germs on the hands of the user. In various embodiments, the beads can be different sizes, and can include different sizes in the same embodiment. The bead-style coating enhancers can be smooth, or can be textured with various textures that can assist in rubbing the sanitizing fluid against the hands of the user. A user can wiggle the hands and/or fingers within the reservoir to create additional movement between the hands and the beads causing the beads to rub between the fingers and against the hands.

FIG. 3B is a perspective view of a thick filament type coating enhancer 310, according to an illustrative embodiment. The thick filament coating enhancer can include a number of flexible thick filaments 312 that can brush against the hands of the user, similar to the brushes of a car wash. By way of non-limiting example, the filaments can be approximately 1-5 mm thick and approximately 5-75 mm long. The filaments 312 can be soft, flexible filaments that can be made from rubber or silicone, or various textile materials, or other materials adapted for brushing against the hands of the user. The walls of the reservoir can be lined with thick filaments 312 that can be attached to, or manufactured as part of the reservoir walls. In various embodiments, the reservoir and the thick filaments 312 can be of unitary construction. As a user inserts hands into the reservoir, the thick filaments can brush the surfaces of the user's hands, thereby assisting the sanitizing fluid in killing germs. Filaments that are attached to the walls of the reservoir can remain in a fixed location, so that they create more fluid coating and/or friction on the hands of the user compared to free-floating coating enhancers within the reservoir when the user pulls the hands through the filaments attached to the reservoir walls.

In various embodiments, the thick filaments 312 can part of free-floating brushers 314. The free-floating brushers can be covered in thick filaments 312. The free-floating brushers 314 can be small objects that may not be attached to the reservoir walls, and can move freely within the reservoir. The filaments on the free-floating brushers can brush the surfaces of the user's hands, thereby assisting the sanitizing fluid in killing germs. The free-floating brushers may be more effective than reservoir-wall filaments at brushing certain surfaces of the hands of the users, such as concave surfaces of the hands.

FIG. 3C is a perspective view of a thin filament type coating enhancer 320, according to an illustrative embodiment. The thin filament type coating enhancer can include a number of flexible rubber strands, or filaments, 322 that can brush against the hands of the user, similar to the brushes of a car wash. By way of non-limiting example, the filaments can be approximately 0.5 to 2 mm thick and approximately 5-75 mm long. The filaments 322 can be soft, flexible filaments that can be made from rubber or silicone, or various textile materials, or other materials adapted for brushing against the hands of the user.

The thin filament type coating enhancer can consist of a number of thin strands of rubber, silicone, textiles, or other materials adapted for brushing against the hands of the user. The walls of the inner reservoir can be lined with thin filaments 322 that can be attached to, or manufactured as part of the reservoir walls. In various embodiments, the reservoir walls and the thin filaments filaments 322 can be of unitary construction. As a user inserts hands into the reservoir, the thin filaments can brush the surfaces of the user's hands, thereby assisting the sanitizing fluid in killing germs. Filaments that are attached to the walls of the reservoir can remain in a fixed location, so that they create more fluid coating and/or friction on the hands of the user compared to free-floating coating enhancers within the reservoir when the user pulls the hands through the filaments attached to the reservoir walls.

In various embodiments, the thin filaments 322 can part of free-floating brushers 324. The free-floating brushers can be made from thin filaments 322 that have been bound together to form balls. The free-floating brushers 324 can be small objects that may not be attached to the reservoir walls, and can move freely within the reservoir. The filaments on the free-floating brushers can brush the surfaces of the user's hands, thereby assisting the sanitizing fluid in killing germs. The free-floating brushers may be more effective than reservoir-wall filaments at brushing certain surfaces of the hands of the users, such as concave surfaces of the hands.

FIG. 3D is a perspective view of a brush-type coating enhancer 330, according to an illustrative embodiment. The brush-type coating enhancer can include a number of bristles 332 that can brush against the hands of the user, similar to the brushes of a car wash. In various embodiments, the reservoir walls and/or free floating brushers can have stiff, or semi-stiff bristles 332. By way of non-limiting example, these various bristles can be similar to the bristles of a toothbrush, the bristles of boar-hair hairbrush, the bristles of a hog-hair paintbrush, the bristles of a fingernail-cleaning brush, or various other brushes used for cleaning various surfaces. Although the many different types of available brushes are not individually described herein, it should be clear that a variety of different brushes can be used.

The walls of the reservoir can be lined with bristles 332 that can be attached to, or manufactured as part of the reservoir walls. In various embodiments, the reservoir and the bristles 332 can be of unitary construction. As a user inserts hands into the reservoir, the bristles can brush the surfaces of the user's hands, thereby assisting the sanitizing fluid in killing germs. Bristles that are attached to the walls of the reservoir can remain in a fixed location, so that they create more fluid coating and/or friction on the hands of the user compared to free-floating coating enhancers within the reservoir when the user pulls the hands through the bristles attached to the reservoir walls.

In various embodiments, the bristles 332 can part of free-floating brushers. The free-floating brushers can be covered in bristles 332. The free-floating brushers can be small objects that may not be attached to the reservoir walls, and can move freely within the reservoir. The bristles on the free-floating brushers can brush the surfaces of the user's hands, thereby assisting the sanitizing fluid in killing germs. The free-floating brushers may be more effective than reservoir-wall brushers at brushing certain surfaces of the hands of the users, such as concave surfaces of the hands.

In various embodiments, small motors may spin or otherwise move the various contact enhancers within the reservoir. This can include spinning motions, back and forth motions, or combinations of motions that can assist with rubbing the sanitizing solution on the hands of the users.

In various embodiments, different combinations of the above coating enhancers are possible, including single coating enhancers that include features from different coating enhancers described herein, and including combinations of different coating enhancers within the same reservoir. By way of non-limiting example, a bead-style coating enhancer can have various filaments extending from the bead, and different beads can have different thicknesses and/or lengths of filaments. As another example, various beads and/or brushers can be present together in the same reservoir. A reservoir may have filaments as part of the reservoir wall and also have one or more forms of free-floating brushers within the reservoir.

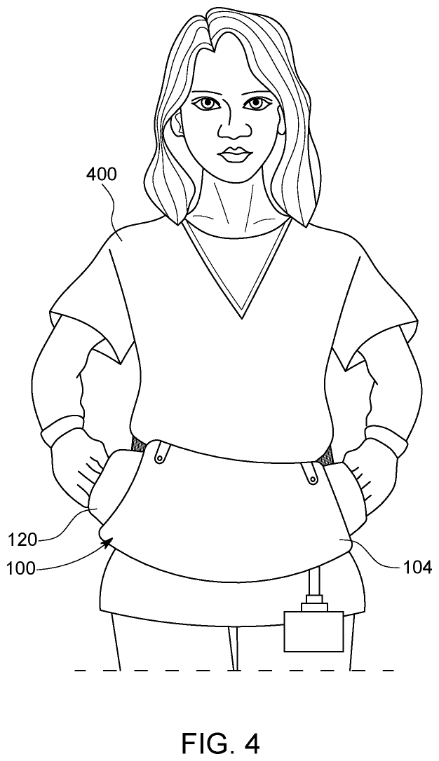

FIG. 4 is a perspective view of a mobile hand sanitizing system worn around the waist of a user, according to an illustrative embodiment. In various embodiments, the mobile hand sanitizing system can have a strap that wraps around the user to secure the mobile hand sanitizing system to the user. In various embodiments, the mobile hand sanitizing system can be incorporated into the clothing of the user, such as incorporation into the front of a set of scrubs. A user 400 can wear the mobile hand sanitizing system in a variety of settings, including healthcare, homecare, daycare, schools, restaurants or other food preparation industries, or other institutions. The strap can hold the hand sanitizing system at a convenient location for the user to insert hands into the body 104 of the hand sanitizing system 100. The user can insert hands into the hand sanitizing system 100, and can wiggle the hands among any coating enhancers within the reservoir. The user can also rub the two hands together, with or without the presence of any coating enhancers. The user can then pull the hands back through the expandable opening of the valve. As the expandable opening stretches around the hands of the user, excess fluid is wiped off of the hands and is retained inside of the mobile hand sanitizing system. As the user pulls the hands out of the mobile hand sanitizing system, the hands can also pass through end cuffs. The end cuffs 120 can be lined with various brushes or wipers to further assist in wiping away excess fluid from the hands of the user. The end cuffs can also include absorbent material that can wipe and absorb excess fluid from the hands of the user. After the user has pulled the hands through the valves, thereby wiping away excess fluid, and then pulled the hands through the end cuffs, thereby further removing remaining fluids, the user's hands can be rid of excess fluid. The user can then rub the two hands together outside of the mobile hand sanitation system to further dry the hands in preparation for the activities to be performed by the user with the freshly sanitized hands.

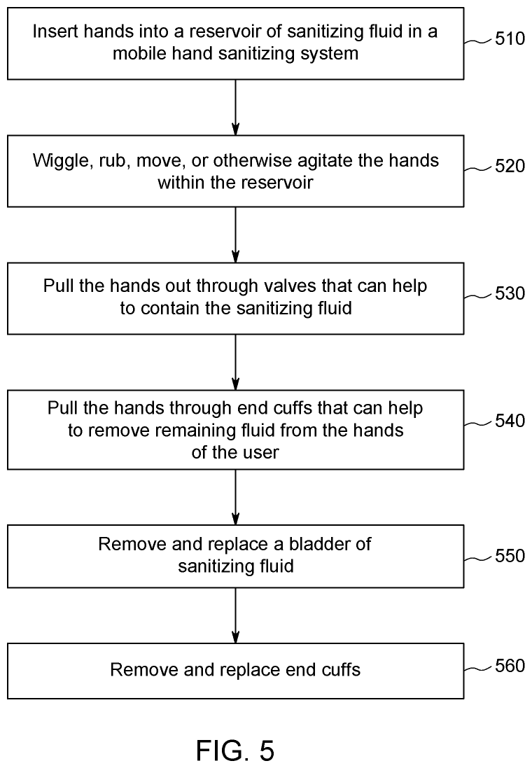

FIG. 5 is a diagram showing a method of mobile hand sanitation 500, according to an illustrative embodiment. At step 510, a user can start by inserting hands into a reservoir of sanitizing fluid within a mobile hand sanitizing system. At step 520, the user can wiggle, rub, move, or otherwise agitate the hands within the reservoir. The hands can be individually rubbed with various coating enhancers and/or the hands can be rubbed together with or without coating enhancers. At step 530, the user can pull the hands out through valves that can help to contain the sanitizing fluid. The valves can include an expandable opening that stretches around the wrists and hands of the user to scrape excess fluid off of the hands and retain the fluid within the reservoir. At step 540, the user can pull the hands through end cuffs. The end cuffs can include various brushes, wipers, and or absorbent materials that can help to remove remaining fluid from the hands of the user. At optional step 550, the user can remove and replace a bladder of sanitizing fluid. At optional step 560, the user can remove and replace end cuffs as they lose the capacity to remove fluid.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, in various embodiments, additional alerts may be present, including alerts to low levels of sanitizing solution. In various embodiments, mechanical systems may be involved in cleaning the hands and/or removing excess fluid from the hands as the hands are removed from the mobile hand sanitizing system. Also, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Accordingly, this description is meant to be taken only by way of example, and not to otherwise limit the scope of this invention.

What is claimed is:

1. A mobile hand sanitizing system comprising:
an outer shell having an opening at each of two ends of the outer shell; and
a reservoir within the outer shell, the reservoir adapted to be filled with a sanitizing fluid.

2. The mobile hand sanitizing system of claim 1, further comprising an end cuff lined with absorbent material, wherein the end cuff is adapted to wipe sanitizing fluid from the hands of the user.

3. The mobile hand sanitizing system of claim 1, further comprising an end cuff lined with brushes, wherein the end cuff is adapted to wipe sanitizing fluid from the hands of the user.

4. The mobile hand sanitizing system of claim 1, further comprising at least one valve at each of the openings, the valves adapted to wipe sanitizing fluid from hands of a user into the reservoir.

5. The mobile hand sanitizing system of claim 1, further comprising one or more coating enhancers within the reservoir, the coating enhancers including various filaments.

6. The mobile hand sanitizing system of claim 1, further comprising a disposable and replaceable bladder, the bladder further comprising a sanitizing solution.

7. The mobile hand sanitizing system of claim 1, further comprising one or more coating enhancers within the reservoir.

8. The mobile hand sanitizing system of claim 7, wherein the coating enhancers further comprise filaments of rubber attached to the sidewalls of the reservoir.

9. The mobile hand sanitizing system of claim 7, wherein the coating enhancers further comprise free-floating balls within the reservoir.

10. The mobile hand sanitizing system of claim 8, wherein the free-floating balls have filaments of rubber.

11. A disposable bladder for use in a mobile hand sanitizing system, the bladder comprising:
an outer sidewall adapted to contain sanitizing fluid;
sanitizing fluid; and
coating enhancers within the disposable bladder.

12. The disposable bladder of claim 11, wherein the coating enhancers include filaments on the bladder walls, the filaments extending into the sanitizing fluid.

13. The disposable bladder of claim 11, wherein the coating enhancers include free-floating balls within the sanitizing fluid.

14. The disposable bladder of claim 13, wherein the free-floating balls have filaments extending outward from the balls.

15. A method of hand sanitization comprises:
inserting hands into a mobile hand sanitizing system, the mobile hand sanitizing system including a reservoir of sanitizing fluid;
moving the hands within the hand sanitizing solution; and
pulling the hands out of the reservoir.

16. The method of claim 15, wherein pulling the hands out of the reservoir further comprises pulling the hands through a valve, the valve adapted to wipe excess fluid off of the hands and retain the fluid within the reservoir.

17. The method of claim 15, further comprising pulling the hands through end cuffs, the end cuffs including absorbent materials adapted to remove fluid from the hands of the user.

18. The method of claim 17, further comprising removing the end cuffs after they become saturated with fluid and replacing the end cuffs with different end cuffs.

19. The method of claim 15, further comprising pulling the hands through end cuffs, the end cuffs including brushes adapted to remove fluid from the hands of the user.
